(19) United States
(12) Patent Application Publication
(10) Pub. No.: US 2024/0042080 A1
(43) Pub. Date: Feb. 8, 2024

(54) A Method And A System For Adjusting A Cycle Time Of A Treatment Process For An Object

(71) Applicant: ASP Global Manufacturing GMBH, Schaffhausen (CH)

(72) Inventor: Benjamin Fryer, Irvine, CA (US)

(21) Appl. No.: 18/268,770

(22) PCT Filed: Dec. 8, 2021
(86) PCT No.: PCT/IB2021/061465

§ 371 Date: Jun. 21, 2023

Related U.S. Application Data
(60) Provisional application No. 63/128,233, filed on Dec. 21, 2020.

(51) Int. Cl. A61L 2/20; A61L 2/24; A61L 2/14

(52) U.S. Cl. CPC A61L 2/208; A61L 2/24; A61L 2/14; A61L 2202/121; A61L 2202/24; A61L 2202/14; A61L 2202/122

(57) ABSTRACT

A method and a system for adjusting a cycle time of a treatment process for an object are provided. The method comprises introducing a treatment agent comprising hydrogen peroxide into a chamber comprising the object and conducting a first treatment stage therein. A hydrogen peroxide vapor concentration in the chamber is measured utilizing a sensor during the first treatment stage to obtain a measured concentration. The measured concentration is compared to a threshold concentration value to obtain a comparison value. A cycle time of the first treatment stage is adjusted based on the comparison value.

### CROSS REFERENCE TO RELATED APPLICATIONS

This application claims priority to U.S. Provisional Patent No. 63/128,233, which was filed on Dec. 21, 2020, and which the contents thereof are hereby incorporated by reference into this specification.

### FIELD

The present disclosure relates to a method and a system for adjusting a cycle time of a treatment process for an object.

### BACKGROUND

Medical devices are used in numerous procedures in the medical field. These devices are as varied as the procedures themselves. As such, proper care of these devices can be critical for efficient, effective, and safe use of the devices during treatment of the patient.

After a medical device is used, the medical device can be disinfected, and/or sterilized in order to prepare the medical device for its next use. This treatment process may include placing the medical device in a treatment device and contacting the medical device with a vapor comprising hydrogen peroxide that disinfects and/or sterilizes the device.

Improving health and safety conditions for patients, as well as improving patient treatment processes are an important focus of the medical field. In this regard, efforts have been made to improve the efficiency and effectiveness of disinfecting, and/or sterilizing processes for medical devices.

### SUMMARY

The present disclosure provides a method for adjusting a cycle time of a treatment process for an object. The method comprises introducing a treatment agent comprising hydrogen peroxide into a chamber comprising the object and conducting a first treatment stage therein. A hydrogen peroxide vapor concentration in the chamber is measured utilizing a sensor during the first treatment stage to obtain a measured concentration. The measured concentration is compared to a threshold concentration value to obtain a comparison value. A cycle time of the first treatment stage is adjusted based on the comparison value.

The present disclosure also provides a system for adjusting a cycle time of a treatment process for an object. The system comprises a chamber, a treatment agent delivery system, a sensor, and a controller. The chamber is configured to receive an object for a first treatment stage therein. The treatment agent delivery system is in fluid communication with the chamber and configured to introduce a treatment agent comprising hydrogen peroxide into the chamber. The sensor is in communication with the chamber and configured to measure a hydrogen peroxide vapor concentration in the chamber during the first treatment stage to obtain a measured concentration. The controller is in signal communication with the sensor. The controller is configured to compare the measured concentration to a threshold concentration to obtain a comparison value and output a control signal in order to adjust a cycle time of the first treatment stage based on the comparison value.

The present disclosure also provides a system comprising a processor coupled to a non-transitory memory. The non-transitory memory comprises machine executable instructions that when executed by the processor cause the processor to introduce a treatment agent comprising hydrogen peroxide into a chamber comprising an object and conduct a first treatment stage therein. Additionally, the machine executable instructions when executed by the processor cause the processor to measure a hydrogen peroxide vapor concentration in the chamber utilizing a sensor during the first treatment stage to obtain a measured concentration and compare the measured concentration to a threshold concentration value to obtain a comparison value. The machine executable instructions when executed by the processor cause the processor to adjust a cycle time of the first treatment stage based on the comparison value.

It is understood that the inventions described in this specification are not limited to the examples summarized in this Summary. Various other aspects are described and exemplified herein.

### BRIEF DESCRIPTION OF THE DRAWINGS

The features and advantages of the examples, and the manner of attaining them, will become more apparent and the examples will be better understood by reference to the following description of examples taken in conjunction with the accompanying drawings, wherein:

FIG. 1 is a block diagram of an example of a system for adjusting a cycle time of a treatment process for an object according to the present disclosure; and

FIG. 2 is a flow chart illustrating an example of a method for adjusting a cycle time of a treatment process for an object according to the present disclosure;

FIG. 3 is a chart illustrating the cumulative AUCs of first half cycles monitored according to the Example described herein; and

FIG. 4 is a chart illustrating the cumulative AUCs of second half cycles monitored according to the Example described herein.

Corresponding reference characters indicate corresponding parts throughout the several views. The exemplifications set out herein illustrate certain examples, in one form, and such exemplifications are not to be construed as limiting the scope of the examples in any manner.

### DETAILED DESCRIPTION

Certain exemplary aspects of the present disclosure will now be described to provide an overall understanding of the principles of the structure, function, manufacture, and use of the devices and methods disclosed herein. One or more examples of these aspects are illustrated in the accompanying drawings. Those of ordinary skill in the art will understand that the devices and methods specifically described herein and illustrated in the accompanying drawings are non-limiting exemplary aspects and that the scope of the various examples of the present disclosure is defined solely by the claims. The features illustrated or described in connection with one exemplary aspect may be combined with the features of other aspects. Such modifications and variations are intended to be included within the scope of the present disclosure.

Any patent, publication, or other disclosure material identified herein is incorporated by reference into this specification in its entirety unless otherwise indicated, but only to the extent that the incorporated material does not conflict with existing descriptions, definitions, statements, or other disclosure material expressly set forth in this specification. As such, and to the extent necessary, the express disclosure as set forth in this specification supersedes any conflicting material incorporated by reference. Any material, or portion thereof, that is said to be incorporated by reference into this specification, but which conflicts with existing definitions, statements, or other disclosure material set forth herein, is only incorporated to the extent that no conflict arises between that incorporated material and the existing disclosure material. Applicants reserve the right to amend this specification to expressly recite any subject matter, or portion thereof, incorporated by reference herein.

Any references herein to “various examples,” “some examples,” “certain examples,” “one example,” “an example,” similar references to “aspects,” or the like, means that a particular feature, structure, or characteristic described in connection with the example is included in at least one example. Thus, appearances of the phrases “in various examples,” “in some examples,” in certain examples,” “in one example,” “in an example,” similar references to “aspects,” or the like, in places throughout the specification are not necessarily all referring to the same example. Furthermore, the particular features, structures, or characteristics may be combined in any suitable manner in one or more examples. Thus, the particular features, structures, or characteristics illustrated or described in connection with one example may be combined, in whole or in part, with the features, structures, or characteristics of one or more other examples without limitation. Such modifications and variations are intended to be included within the scope of the present examples.

In this specification, unless otherwise indicated, all numerical parameters are to be understood as being prefaced and modified in all instances by the term “about”, in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Also, any numerical range recited herein includes all sub-ranges subsumed within the recited range. For example, a range of “1 to 10” includes all sub-ranges between (and including) the recited minimum value of 1 and the recited maximum value of 10, that is, having a minimum value equal to or greater than 1 and a maximum value equal to or less than 10. Any maximum numerical limitation recited in this specification is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited.

The grammatical articles “a,” “an,” and “the,” as used herein, are intended to include “at least one” or “one or more,” unless otherwise indicated, even if “at least one” or “one or more” is expressly used in certain instances. Thus, the articles are used herein to refer to one or more than one (i.e., to “at least one”) of the grammatical objects of the article. Further, the use of a singular noun includes the plural, and the use of a plural noun includes the singular, unless the context of the usage requires otherwise.

A device can undergo a treatment process to prevent cross-contamination and the spread of disease. As used herein, a “treatment process” may be a disinfecting process, a sterilization process, the like, and combinations thereof. As used herein, a “treatment agent” can comprise at least one of a disinfectant and a sterilant. For example, the treatment agent can comprise hydrogen peroxide. A disinfecting process and a sterilization process can remove and/or eliminate a bioburden from an object. A bioburden may be, for example, a bacterium (e.g., mycobacterium, bacterial spores), an archaeon, a eukaryote, a virus, a fungus, and/or other forms of biological agents. Bacterial spores (e.g., endospores) are a form of bacteria which are dormant and highly resistive to physical and chemical degradation. As used herein, a “disinfecting process” means a treatment process that substantially removes a bioburden except for bacterial spores. As used herein, “substantially remove” means that at least 99% of the bioburden has been removed from the object such as, for example, at least 99.9% of the bioburden, at least 99.99% of the bioburden, at least 99.999% of the bioburden, or at least 99.9999% of the bioburden has been removed from the object. The disinfection process may include, for example, the addition of heat, a disinfectant, irradiation, pressure, and combinations thereof. The disinfectant may comprise a chemical capable of disinfection. As used herein, a “sterilization process” means a treatment process which substantially removes a bioburden including bacterial spores. The sterilization process may include, for example, the addition of heat, freezing, a sterilant, irradiation, pressure, and combinations thereof. The sterilant may comprise a chemical capable of sterilization.

A treatment process for a device, such as, for example, a medical device, can comprise various processing steps. For example, the treatment process can comprise bulk removal and/or elimination of debris from the device and removal and/or elimination of bioburden from the device. Each device can have varying amounts of debris and/or bioburden which can affect the treatment process. For example, use of a medical device on a patient can result in a device that can be soiled to various levels, depending on the medical procedure(s) that are performed. Manufacturing the medical device can also soil the device to various levels.

In various examples, where the treatment process is a sterilization process, a solution of hydrogen peroxide and water (e.g., 59% hydrogen peroxide by weight) can be introduced to the treatment apparatus and vaporized into a treatment chamber. The solution can contact an object within the treatment chamber and create a biocidal environment that can inactivate bioburden with chemical interactions at multiple biologically important reaction sites. A strong electrical field can then be applied to the treatment chamber that can create a hydrogen peroxide gas plasma that dissociates hydrogen peroxide molecules into energized species. After the electrical field is turned off, the energized species can recombine, which converts the hydrogen peroxide into water and oxygen.

Typically, sterilization processes are designed with fixed cycle times that can ensure the maximum validated loading of the treatment chamber is sterilized (e.g., maximum bioburden that can be removed). This can be a conservative approach to ensure that the challenging loads are sterilized. The present inventors have determined that many of the sterilization processes are not processing challenging loads, and the processed loads can be significantly less challenging, and may be, for example, approximately half or less as challenging as the maximum validation load. Using a fixed cycle time to sterilize the challenging load, when the loads are frequently not that challenging, can be inefficient and/or cause unnecessarily excessive wear to objects to be sterilized.

Thus, provided herein is a method and a system for adjusting a cycle time of a treatment stage for an object. Adjusting the cycle time can increase the efficiency of the treatment process and limit wear to objects in a treatment process. For example, the cycle time of a treatment process can be reduced thereby increasing the throughput in the treatment apparatus.

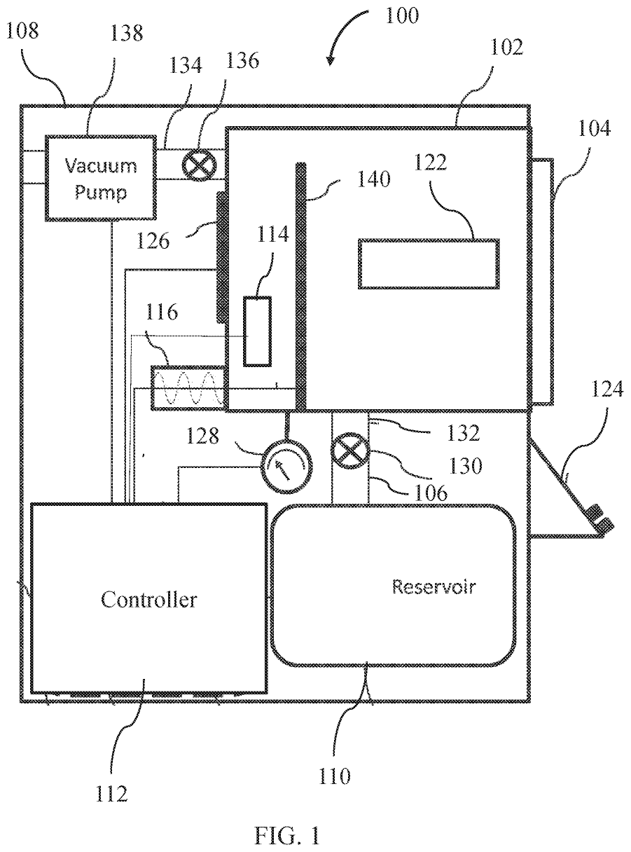

Referring to FIG. 1, a system 100 for adjusting a cycle time of a treatment process for an object in a treatment apparatus 108 is provided. The system 100 can comprise the treatment apparatus 108. The treatment apparatus 108 can be, for example a disinfection system, a sterilization system, or a combination thereof. For example, the treatment apparatus 108 can be a sterilization system that utilizes hydrogen peroxide, such as, for example, a STERRAD® system manufactured by Advanced Sterilization Products, Irvine, California.

The treatment apparatus 108 can comprise a treatment chamber 102, a door 104, a treatment agent source, such as a reservoir 110 as illustrated, a treatment agent delivery system 106, a sensor 114 for determining a hydrogen peroxide vapor concentration in the treatment apparatus 108, and a controller 112. The treatment apparatus 108 can comprise a plasma generator 116 in electromagnetic communication with the treatment chamber 102. In certain examples, the treatment apparatus 108 can comprise a user interface 124, that may include output devices, such as a printer or display, and user-input devices, such as a keypad or touch screen.

The treatment chamber 102 can be formed of any suitable material that can withstand operational pressures in the treatment chamber 102, such as, for example, a pressure as low as 0.3 torr. Additionally, the treatment chamber can be formed of suitable material, which inhibits reacting with and/or absorbing a treatment agent (e.g., hydrogen peroxide) introduced therein. For example, the treatment chamber 102 can comprise aluminum, an aluminum alloy, iron, or an iron alloy (e.g., stainless steel).

The treatment chamber 102 can be configured to receive an object 122, such as a medical device, for a treatment process therein. The object 122 can comprise a medical device, such as, for example, at least one of forceps, a clamp, a retractor, a cutter, a dilator, a tube, a fitting, a stapler, a needle, a drill, a scope, an endoscope, and a probe. For example, the object 122 can be a load (e.g., pack) of medical devices to be treated in the treatment chamber 102.

The treatment chamber 102 can comprise a volume and a shape suitable to receive the object 122. The treatment chamber 102 can be configured to promote contact between a surface of the object 122 and a fluid. The treatment chamber 102 can be configured in a manner that directs the fluid towards the object 122 such that the fluid can flow onto a surface of the object 122.

The treatment agent delivery system 106 can be in fluid communication with the treatment chamber 102 and configured to introduce fluid into the treatment chamber 102. The treatment agent delivery system 106 can also be in communication with the reservoir 110. The treatment agent delivery system 106 can comprise at least one of a pump, a nozzle, a vaporizer, a scrubbing apparatus, a tube, and a fitting and can be in fluid communication with the treatment chamber 102 to transport fluid from the reservoir 110 into the treatment chamber 102 and/or onto a surface of the object 122. For example, the treatment agent delivery system 106 can comprise a hose or pipe 130 connected to the treatment chamber 102 and a valve 132 can be disposed between the treatment chamber 102 and the reservoir 110 to control the flow of fluid from reservoir 110 through the hose or pipe 130 and into treatment chamber 102. The fluid that enters the chamber 102 comprises gas and minimally, if any liquid. Liquid water may be removed from the fluid in an evaporator/condenser unit thereby condensing hydrogen peroxide to obtain at least 85% hydrogen peroxide. The condensed hydrogen peroxide can then be vaporized and introduced into the chamber. This method, however, is applicable to other sterilant gases, such as, for example, peracetic acid, acetic acid, and nitric acid

Fluid can be dispensed from the treatment agent delivery system 106 into contact with an outer and/or internal surface of the object 122. The fluid can be any suitable fluid, such as one for disinfecting and/or sterilizing the surface of the object 122 and can comprise a treatment agent comprising hydrogen peroxide and optionally, water, solvent, and/or other reagent. The fluid can comprise hydrogen peroxide and water. For example, the fluid can comprise at least 1% hydrogen peroxide by weight, such as, for example, at least 10% hydrogen peroxide by weight, at least 20% hydrogen peroxide by weight, at least 30% hydrogen peroxide by weight, at least 40% hydrogen peroxide by weight, or at least 50% hydrogen peroxide by weight. The fluid can be introduced to the treatment chamber 102 as a liquid, a gas, a mist, a vapor, a gaseous composition, or a combination thereof. The fluid can be introduced into the treatment chamber as a gas.

As used herein, a “mist” is meant to mean a substance comprising small droplets of liquid that are suspended in a gas. Mist can vaporize or evaporate into vapor. Mist may not condense, as mist is already in the liquid phase. Mist can be generated with a suitable liquid droplet generating device such as, for example, an ultrasound humidifier. Depending on the size and density of the small droplets of liquid, mist is generally visible to the naked eye.

As used herein, a “vapor” is meant to mean a substance in the gas phase that has a temperature lower than the critical temperature of the substance such that the vapor can be condensed to a liquid by increasing the pressure without reducing the temperature. Vapor can condense into a liquid phase from the gas phase. In various examples, vapor is distinct from mist.

A “gaseous composition” as used herein is meant to mean a liquid, a gas, or a combination thereof such as, for example, a vapor, a mist, a gas that has a temperature at least the critical temperature of the substance, or a combination thereof. For example, a gaseous water composition can comprise water vapor, a water mist, water gas, or combinations thereof.

The door 104 can be a sealable barrier positionable in a first configuration and a second configuration. The first configuration can enable access to the treatment chamber 102 for loading and/or unloading of the object 122, such as in a generally “open” position. The second configuration can limit access to the treatment chamber 102 and can inhibit excess fluid from leaving the treatment chamber 102. For example, in the second configuration, the door 104 may be positioned in a sealable arrangement with the treatment chamber 102 to inhibit access to the treatment chamber 102 during the treatment process, such as in a generally “closed” position. The door 104 can be configured to withstand operational pressure within the treatment chamber 102 and limit leaks between chamber 102 and the ambient environment.

The treatment apparatus 108 can comprise a pressure device, such as a vacuum pump 138 as illustrated, configured to adjust and/or maintain a pressure within the treatment chamber 102. For example, the vacuum pump 138 can remove air and other gases, such as water vapor, from the treatment chamber 102. A hose or pipe 134 can be connected to the vacuum pump 138 and the treatment chamber 102. The vacuum pump 138 may also include a valve 136 that may be opened or closed to control the pressure in the treatment chamber 102. For example, when the valve 136 is open and the vacuum pump 138 is operational, the pressure in the treatment chamber 102 can be lowered. In certain examples, when the valve 136 is open and the vacuum pump 138 is not operational, the pressure in the treatment chamber 102 may be equalized to the ambient pressure. The valve 136 may be separate from the vacuum pump 138. The treatment apparatus 108 can comprise a pressure monitor 128 configured to monitor the pressure in the treatment chamber 102. For example, the pressure monitor 128 can be a capacitance manometer available from MKS Instruments.

The treatment apparatus 108 can comprise a temperature control unit, such as a heating element 126 as illustrated, configured to control the temperature in the treatment chamber 102. The heating element 126 can comprise separate elements bonded to the outside of the chamber 102 in locations sufficient to uniformly heat the treatment chamber 102.

The reservoir 110 can comprise fluid, such as, for example, hydrogen peroxide and optionally, water, solvent, and/or other reagent. There may be a plurality of reservoirs in fluid communication with the treatment chamber 102 and each reservoir can comprise at least one of a hydrogen peroxide, water, a solvent, and a reagent. For example, a reservoir can comprise a cartridge that contains a plurality of capsules and each capsule can comprise the fluid. In various examples, the fluid within one or at least two capsules can be per cycle. The sensor 114 can be in direct and/or indirect fluid communication with the treatment chamber 102 and configured to measure a hydrogen peroxide vapor concentration in the treatment chamber 102 during the first treatment stage to obtain a measured concentration. For example, the sensor 114 may be positioned within the treatment chamber 102. In various examples, the treatment apparatus 108 can comprise a port (e.g., tube, window, wall) through which the sensor 114 can emit and/or receive electromagnetic radiation and/or other signal.

The sensor 114 can comprise an electrochemical sensor, a photolysis sensor, a photometric sensor, a metal-oxide sensor, or a combination thereof. The sensor 114 can comprise a hydrogen peroxide vapor concentration sensor. The sensor 114 can be an ultra-violent sensor, such as, for example, an ultraviolet sensor that operates with a center wavelength of 254 nm.

The sensor 114 can be in signal communication with the controller 112. For example, the sensor 114 can communicate with the controller 112 via electrical signals (e.g., electrical impulses) that can be received by and/or emitted by the controller 112. The sensor 114 can communicate with the controller 112 via a wire or a wireless communication technology (e.g., Bluetooth, WiFi).

The controller 112 can be configured to compare the measured concentration to a threshold concentration to obtain a comparison value. For example, the controller 112 can be configured to determine if the measured concentration meets or exceeds the threshold concentration value. The controller 112 can output a control signal in order to adjust a cycle time of the first treatment stage based on the comparison value based on the comparison. For example, the controller 112 can be configured to reduce the cycle time of the first treatment stage if the measured concentration meets or exceeds the threshold concentration value.

The controller 112 can communicate with the treatment apparatus 108 in order to control the introduction of fluid into the treatment chamber 102 by the treatment agent delivery system 106 and/or generation of a plasma by the plasma generator 116. The controller 112 can be configured to adjust a parameter of a second treatment stage based on the adjusted cycle time of the first treatment stage. For example, the controller 112 can be configured to reduce a cycle time of the second treatment stage based on the reduced cycle time of the first treatment stage.

The plasma generator 116 can be configured to expose the object 122 to a plasma in the second treatment stage. The plasma can facilitate disinfecting and/or sterilization and remediate treatment agent in the treatment chamber 102. For example, the plasma generator 116 can comprise a power source and/or signal generator and an electrode 140 disposed within chamber 102, which are configured to create an electric field within the treatment chamber 102 between the electrode 140 and an interior surface of the treatment chamber 102 to create a plasma therein. A signal, such as an RF signal, may be provided to electrode 140 from power source and/or signal generator by way of a feed through, such as a wire-type feed through. Creation of a plasma can be useful for low temperature sterilization processes that use hydrogen peroxide vapor. In these processes, the hydrogen peroxide vapor may be excited to form a hydrogen peroxide plasma. In various examples, another gas and/or vapor may be used to form the plasma, such as air, which may help lower hydrogen peroxide residuals upon the load to facilitate removal of hydrogen peroxide from the treatment chamber 102.

The system 100 can comprise a second sensor (not shown) configured to measure a second parameter in the treatment chamber 102 during the first treatment stage to obtain a measured second parameter. The second sensor can be in signal communication with the controller 112. The second sensor can be a pressure sensor in fluid communication with the treatment chamber 102 and the second parameter can be a pressure in the treatment chamber 102 during the first treatment stage. The controller 112 can be configured to adjust the cycle time of the first treatment stage based on the measured concentration and the measured second parameter.

The controller 112 can comprise a processor operatively coupled to non-transitory memory. The non-transitory memory of the controller 112 can comprise machine executable instructions that when executed by the processor can cause the processor to perform the functions of adjusting a cycle time of a treatment process for the object 122 as described herein. For example, the machine executable instructions when executed by the processor can cause the processor to introduce a treatment agent comprising hydrogen peroxide into the treatment chamber 102 comprising the object 122 and conduct a first treatment stage therein. The machine executable instructions when executed by the processor can cause the processor to measure a hydrogen peroxide vapor concentration in the treatment chamber 102 utilizing the sensor 114 during the first treatment stage to obtain a measured concentration. The machine executable instructions when executed by the processor can cause the processor to compare the measured concentration to a threshold concentration value to obtain a comparison value and adjust a cycle time of the first treatment stage based on the comparison value.

The non-transitory memory of the controller 112 can comprise any machine-readable or computer-readable media capable of storing data, including both volatile and non-volatile memory. For example, the non-transitory memory may include read-only memory (ROM), random-access memory (RAM), dynamic RAM (DRAM), Double-Data-Rate DRAM (DDR-RAM), synchronous DRAM (SDRAM), static RAM (SRAM), programmable ROM (PROM), erasable programmable ROM (EPROM), electrically erasable programmable ROM (EEPROM), flash memory (e.g., NOR or NAND flash memory), content addressable memory (CAM), polymer memory (e.g., ferroelectric polymer memory), phase-change memory (e.g., ovonic memory), ferroelectric memory, silicon-oxide-nitride-oxide-silicon (SONOS) memory, disk memory (e.g., floppy disk, hard drive, optical disk, magnetic disk), or card (e.g., magnetic card, optical card), or any other type of media suitable for storing information.

The processor of the controller 112 can be a central processing unit (CPU). The processor may be implemented as a general purpose processor, a chip multiprocessor (CMP), a dedicated processor, an embedded processor, a digital signal processor (DSP), a network processor, a media processor, an input/output (I/O) processor, a media access control (MAC) processor, a radio baseband processor, a vector co-processor, a microprocessor such as a complex instruction set computer (CISC) microprocessor, a reduced instruction set computing (RISC) microprocessor, and/or a very long instruction word (VLIW) microprocessor, or other processing device. The processor also may be implemented by a microcontroller, an application specific integrated circuit (ASIC), a field programmable gate array (FPGA), a programmable logic device (PLD), and so forth. The processor can be configured to run an operating system (OS) and various other applications.

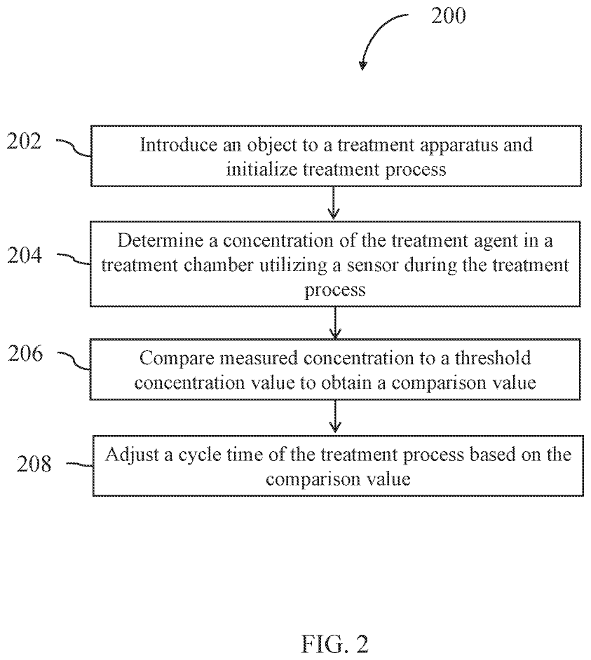

Referring to FIG. 2, a flow chart 200 is provided that illustrates an example of a method for adjusting a cycle time of a treatment process for an object according to the present disclosure. For example, the object 122 to be treated can be introduced into the treatment apparatus 108 and the treatment process can be initialized, 202. Introducing the object 122 to the treatment apparatus 108 can comprise positioning the object in the treatment chamber 102 of the treatment apparatus 108 and using the door 104 to seal the object 122 within the treatment chamber 102.

A treatment agent comprising hydrogen peroxide can be introduced into the treatment chamber 102 and a first treatment stage of the treatment process can be conducted therein. The first treatment stage can comprise contacting a surface of the object 122 with hydrogen peroxide vapor. Contacting the surface of the object 122 can be by various means and can comprise, for example, at flowing the hydrogen peroxide vapor onto a surface of the object 122. The treatment agent can be introduced to the treatment chamber 102 by introducing a fluid comprising the treatment agent to the treatment chamber 102.

The method of the present disclosure can further comprise determining the hydrogen peroxide vapor concentration in the treatment chamber 102 utilizing the sensor 114 during the treatment process to obtain a measured concentration, 204. For example, the hydrogen peroxide vapor concentration can be measured during a first treatment stage of the treatment process. The hydrogen peroxide vapor concentration can be a molar concentration per unit volume, a mass per unit volume concentration, a volume per unit volume concentration, a mass per unit area concentration, variations thereof, and combinations thereof. Evaluating the hydrogen peroxide vapor concentration in the treatment chamber 102 can aid in performance of a treatment process for the object 122. For example, a first object comprising a higher amount of bioburden can be more difficult to disinfect and/or sterilize than a second object comprising a lower amount of bioburden, and require, for example, a longer processing time.

The hydrogen peroxide vapor concentration can be determined over a period of time and an area under the curve (AUC) value can be determined from the concentration over the period of time. Thus, the cumulative effect of the hydrogen peroxide vapor on the object 122 can be determined and utilized to determine the efficacy of the treatment. The cumulative effect of the hydrogen peroxide vapor on the object 122 during the treatment process can be determined by determining the definite integral of the measured concentrations during the portion of the first treatment cycle that has elapsed.

A second parameter of the first treatment stage can be measured to obtain a measured second parameter. The second parameter can be a pressure in the treatment chamber during the first treatment stage or other parameter. Similar to the measured concentration, the second parameter can be determined over a period of time and a second AUC value can be determined from the second parameter over the period of time.

The measured concentration can be compare to a threshold concentration value to obtain a comparison value, 206. The threshold concentration value can be a minimal AUC value required for efficacy for the treatment process (e.g., based on prior research, based on standards) plus a safety margin. For example, the threshold concentration value can be at least the minimal AUC, such as, for example, at least 1.1 times the minimal AUC, at least 1.5 times the minimal AUC, at least 2 times the minimal AUC, or at least 3 times the minimal AUC. For example, the threshold concentration value can be in a range of the minimal AUC to 4 times the minimal AUC. The minimal AUC can be at least 100 mg hydrogen peroxide-seconds per liter, such as, for example, at least 200 mg hydrogen peroxide-seconds per liter, at least 300 mg hydrogen peroxide-seconds per liter, at least 400 mg hydrogen peroxide-seconds per liter, at least 500 mg treatment agent-second per liter, at least 600 mg treatment agent-second per liter, at least 700 mg hydrogen peroxide-second per liter, at least 747 mg hydrogen peroxide-second per liter, at least 750 mg hydrogen peroxide-second per liter, at least 800 mg hydrogen peroxide-second per liter, or at least 900 mg hydrogen peroxide-second per liter. For example, the minimal AUC can be in a range of 100 mg hydrogen peroxide-second per liter to 2000 mg hydrogen peroxide-second per liter, such as, for example, 500 mg hydrogen peroxide-second per liter to 1500 mg hydrogen peroxide-second per liter or 600 mg hydrogen peroxide-second per liter to 900 mg hydrogen peroxide-second per liter.

Comparing the measured concentration to the threshold concentration value can comprise determining if the measured concentration meets or exceeds the threshold concentration value. For example, the cumulative effect of the hydrogen peroxide vapor on the object 122 can be compared to the threshold concentration value to determine if the cumulative effect of the hydrogen peroxide vapor on the object 122 meets or exceeds the threshold concentration value. Accordingly, the comparison can determine if the minimal AUC value required for efficacy for the treatment process has been reached.

A cycle time of the treatment process can be adjusted (e.g., increased, decreased) based on the comparison value, 208. For example, the cycle time of the first treatment stage can be adjusted. The cycle time of the first treatment stage can be reduced based on the comparison if the measured concentration meets or exceeds the threshold concentration value. Conversely, the cycle time of the first treatment stage can be increased or maintained based on the comparison if the measured concentration does not meet or exceed the threshold concentration value. The definite integral of the measured concentration during the portion of the first treatment cycle that has elapsed can be dynamically determined and the additional time required to reach the threshold concentration value can be predicted. Based on the predicted time to reach the threshold concentration value, the cycle time of the first treatment stage can be adjusted. For example, the definite integral of the measured concentration during the portion of the first treatment cycle that has elapsed can be dynamically determined and when the definite integral meets or exceeds the threshold concentration value, the first treatment stage can be stopped and/or the second treatment stage can be initialized. The cycle time of the first treatment stage can be adjusted based on both the measured concentration and the measured second parameter. Adjusting the cycle time can increase efficiency and/or increase the effectiveness of the treatment process. For example, a treatment process can be completed in less time and/or with less wear on the object than in an unadjusted treatment process.

A parameter of a second treatment stage of the treatment process can be adjusted based on the adjusted cycle time of the first treatment stage. For example, the cycle time of the second treatment stage can be reduced based on the reduced cycle time of the first treatment stage. The second treatment stage can comprise exposing the object to a plasma. The treatment process can comprise a third treatment stage which can be adjusted based on the adjusted cycle time in the first treatment stage.

Adjustment of the cycle time and/or parameter of the second treatment stage (and/or subsequent treatment stage) can be executed automatically, based on the measured concentration. Concentration measurements can be determined from a single location or a plurality of locations within the treatment chamber 102, and the measured concentrations from the plurality of locations can each be considered alone or in combination (e.g., averaged, weighted) when adjusting the treatment process. The determination of the measured concentrations can verify successful completion of a treatment process. For example, if the measured concentration is determined to meet or exceed a threshold concentration value, the treatment process could be verified as successfully completed.

### Example

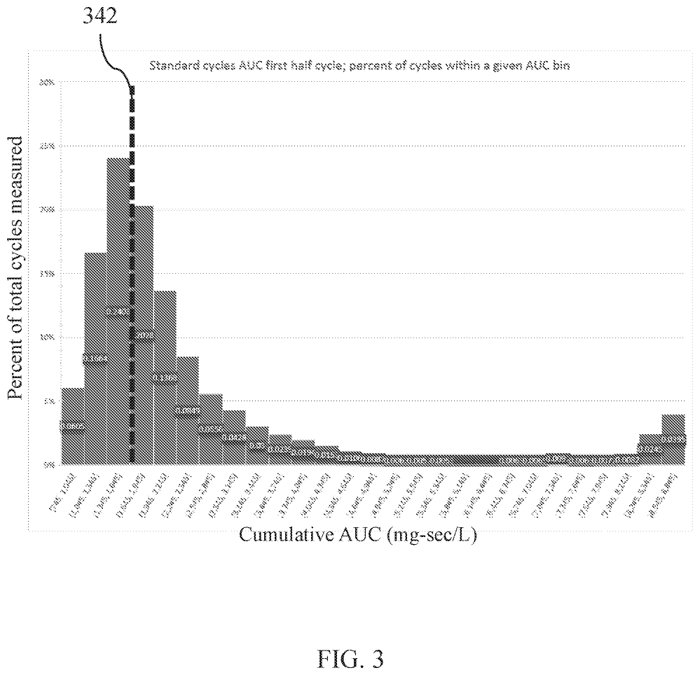

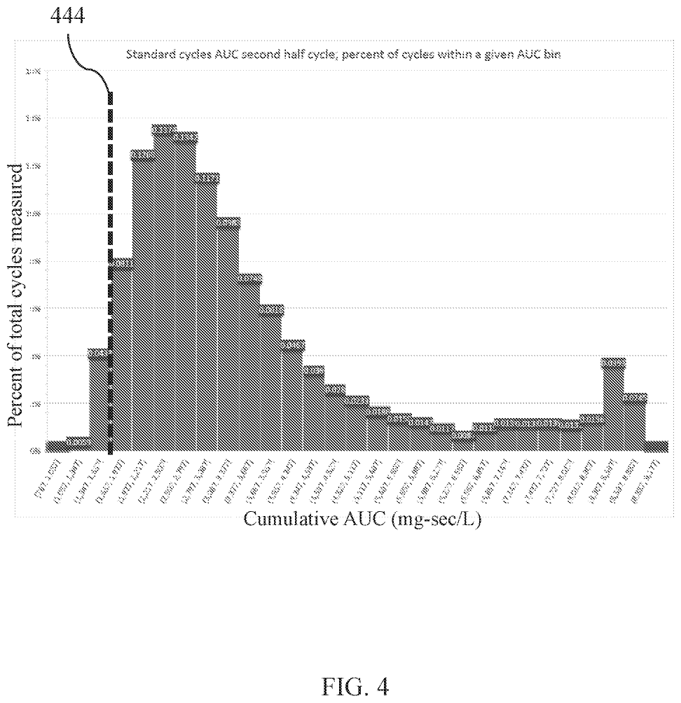

In order to determine the increase in efficiencies and time saving that can be achieved utilizing the methods and systems according to the present disclosure, over various sterilization cycles in STERRAD® sterilization system from Advanced Sterilization Products, Irvine, California were monitored for hydrogen peroxide vapor concentration. For each cycle, the STERRAD® sterilization system injects hydrogen peroxide vapor into a chamber comprising various objects with various soil levels for sterilization in two different stages, a first half cycle and a second half cycle. The hydrogen peroxide concentration was measured every 1 seconds in each cycle utilizing a ultraviolet sensor over the total cycle time of 8 minutes. A cumulative AUC value of each half cycle was determined for each sterilization cycle. The cumulative AUC of each first half cycle was binned in FIG. 3 and the cumulative AUC of each second half cycle was binned in FIG. 4.

As shown in FIG. 3, if a threshold concentration value of 1645 mg-sec/L (e.g., 747 mg-sec/L with a ˜2.2 safety margin) was preset as shown by line 342 in FIG. 3, 56% of cycles could have reduced the cycle time of the first half of the cycle and/or could have skipped the second half of the cycle (e.g., second hydrogen peroxide vapor injection). Additionally, as shown by line 444 in FIG. 4, 95% of the cycles could have had reduced the cycle time of the second half of the cycle.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

One skilled in the art will recognize that the herein described components, devices, operations/actions, and objects, and the discussion accompanying them, are used as examples for the sake of conceptual clarity and that various configuration modifications are contemplated. Consequently, as used herein, the specific examples/embodiments set forth and the accompanying discussion are intended to be representative of their more general classes. In general, use of any specific exemplar is intended to be representative of its class, and the non-inclusion of specific components, devices, operations/actions, and objects should not be taken limiting. While the present disclosure provides descriptions of various specific aspects for the purpose of illustrating various aspects of the present disclosure and/or its potential applications, it is understood that variations and modifications will occur to those skilled in the art. Accordingly, the invention or inventions described herein should be understood to be at least as broad as they are claimed and not as more narrowly defined by particular illustrative aspects provided herein.

1. A method for adjusting a cycle time of a treatment process for an object, the method comprising:
introducing a treatment agent comprising hydrogen peroxide into a chamber comprising the object and conducting a first treatment stage therein;
measuring a hydrogen peroxide vapor concentration in the chamber utilizing a sensor during the first treatment stage to obtain a measured concentration;
comparing the measured concentration to a threshold concentration value to obtain a comparison value; and
adjusting a cycle time of the first treatment stage based on the comparison value.

2. The method of claim 1, wherein the sensor comprises an electrochemical sensor, a photolysis sensor, a photometric sensor, a metal-oxide sensor, or a combination thereof.

3. The method of claim 1, wherein:
the comparing the measured concentration to the threshold concentration value comprises determining if the measured concentration meets or exceeds the threshold concentration value, and
the adjusting the cycle time of the first treatment stage based on the comparison comprises reducing the cycle time of the first treatment stage if the measured concentration meets or exceeds the threshold concentration value.

4. The method of claim 1, further comprising adjusting a parameter of a second treatment stage based on the adjusted cycle time of the first treatment stage.

5. The method of claim 4, wherein the second treatment stage comprises exposing the object to a plasma.

6. The method of claim 4, wherein the adjusting the parameter of the second treatment stage comprises reducing a cycle time of the second treatment stage based on the reduced cycle time of the first treatment stage.

7. The method of claim 1, further comprising measuring a second parameter of the first treatment stage to obtain a measured second parameter, wherein the cycle time of the first treatment stage is adjusted based on the measured concentration and the measured second parameter.

8. The method of claim 7, wherein the second parameter is a pressure in the chamber during the first treatment stage.

9. The method of claim 1, wherein the method is performed in a sterilization system that comprises the chamber and the object is a medical device.

10. A system for adjusting a cycle time of a treatment process for an object, the system comprising:
a chamber configured to receive an object for a first treatment stage therein;
a treatment agent delivery system in fluid communication with the chamber and configured to introduce a treatment agent comprising hydrogen peroxide into the chamber;
a sensor in communication with the chamber and configured to measure a hydrogen peroxide vapor concentration in the chamber during the first treatment stage to obtain a measured concentration; and
a controller in signal communication with the sensor, the controller configured to:
compare the measured concentration to a threshold concentration to obtain a comparison value; and
output a control signal in order to adjust a cycle time of the first treatment stage based on the comparison value.

11. The system of claim 10, wherein the sensor comprises an electrochemical sensor, a photolysis sensor, a photometric sensor, a metal-oxide sensor, or a combination thereof.

12. The system of claim 10, wherein
the controller configured to compare the measured concentration to the threshold concentration value comprises the controller configured to determine if the measured concentration meets or exceeds the threshold concentration value; and
the controller configured to adjust the cycle time of the first treatment stage based on the comparison value comprises the controller configured to reduce the cycle time of the first treatment stage if the measured concentration meets or exceeds the threshold concentration value.

13. The system of claim 10, wherein the controller is configured to adjust a parameter of a second treatment stage based on the adjusted cycle time of the first treatment stage.

14. The system of claim 13, further comprising a plasma generator configured to expose the object to a plasma in the second treatment stage.

15. The system of claim 13, wherein the controller is configured to reduce a cycle time of the second treatment stage based on the reduced cycle time of the first treatment stage.

16.1. The system of claim 13,
further comprising a second sensor configured to measure a second parameter in the chamber during the first treatment stage to obtain a measured second parameter, the second sensor in signal communication with the controller, and
wherein the controller is configured to adjust the cycle time of the first treatment stage based on the measured concentration and the measured second parameter, wherein the second sensor is a pressure sensor and the second parameter is a pressure in the chamber during the first treatment stage.

16.2. (canceled)

17. The system of claim 10, wherein the system comprises a sterilization system and the object is a medical device.

18. A system comprising a processor coupled to a non-transitory memory, wherein the non-transitory memory comprises machine executable instructions that when executed by the processor cause the processor to:
introduce a treatment agent comprising hydrogen peroxide into a chamber comprising an object and conduct a first treatment stage therein;
measure a hydrogen peroxide vapor concentration in the chamber utilizing a sensor during the first treatment stage to obtain a measured concentration;
compare the measured concentration to a threshold concentration value to obtain a comparison value; and
adjust a cycle time of the first treatment stage based on the comparison value.

19. The system of claim 18,
wherein the machine executable instructions that when executed by the processor cause the processor to compare the measured concentration to the threshold concentration value comprises machine executable instructions that when executed by the processor cause the processor to determine if the measured concentration meets or exceeds the threshold concentration value; and
wherein the machine executable instructions that when executed by the processor cause the processor to adjust the cycle time of the first treatment stage based on the comparison value comprises the controller configured to reduce the cycle time of the first treatment stage if the measured concentration meets or exceeds the threshold concentration value.

20. The system of claim 18, wherein the system comprises a sterilization system and the object is a medical device.
